(19) United States
(12) Patent Application Publication
(10) Pub. No.: US 2007/0173538 A1
(43) Pub. Date: Jul. 26, 2007

(54) PHARMACEUTICAL FORMULATION FOR DELIVERY OF RECEPTOR TYROSINE KINASE INHIBITING (RTKi) COMPOUNDS TO THE EYE

(75) Inventors: Wesley Wehsin Han, Arlington, TX (US); Rajni Jani, Fort Worth, TX (US); Huixiang Zhang, Forth Worth, TX (US)

Correspondence Address: ALCON, IP LEGAL, TB4-8, 6201 SOUTH FREEWAY, FORT WORTH, TX, 76134, US

(73) Assignee: Alcon, Inc., Hunenberg (CH)

(21) Appl. No.: 11/613,257

(22) Filed: Dec. 20, 2006

Related U.S. Application Data
(60) Provisional application No. 60/753,749, filed on Dec. 23, 2005.

(51) Int. Cl. A61K 31/416; A61K 31/08

(52) U.S. Cl. 514/406; 514/723

(57) ABSTRACT

The present invention relates to development of efficacious pharmaceutical compositions comprising a poorly water soluble active compound in a therapeutically effective amount and a co-solvent in a suitable amount to treat or prevent diseases due to ocular neovascularization and enhanced vascular permeability. In preferred aspects the composition is in the form of a gel.

This application claims priority to U.S. provisional application Ser. No. 60/753,749 filed Dec. 23, 2005.

### BACKGROUND OF THE INVENTION

1. Field of the Invention

The present invention relates to compositions and methods useful for treating pathological states that arise or are exacerbated by ocular angiogenesis and vascular leakage such as AMD, DR, diabetic macular edema etc., and more specifically, to compositions containing at least one anti-angiogenic agent, anti-inflammatory agent, or anti-vascular permeability agent for use in treating angiogenic ocular disorders.

2. Description of the Related Art

Abnormal neovascularization or angiogenesis and enhanced vascular permeability are major causes for many ocular disorders including age-related macular degeneration (AMD), retinopathy of prematurity (ROP), ischemic retinal vein occlusions and diabetic retinopathy (DR). AMD and DR are among the most common cause of severe, irreversible vision loss. In these and related diseases, such as retinal vein occlusion, central vision loss is secondary to angiogenesis, the development of new blood vessels from pre-existing vasculature, and alterations in vascular permeability properties.

The angiogenic process is known by the activation of quiescent endothelial cells in pre-existing blood vessels. The normal retinal circulation is resistant to neovascular stimuli, and very little endothelial cell proliferation takes place in the retinal vessels. While there appear to be many stimuli for retinal neovascularization, including tissue hypoxia, inflammatory cell infiltration and penetration barrier breakdown, all increase the local concentration of cytokines (VEGF, PDGF, FGF, TNF, IGF etc.), integrins and proteinases resulting in the formation of new vessels, which then disrupt the organizational structure of the neural retina or break through the inner limiting membranes into the vitreous. Elevated cytokine levels can also disrupt endothelial cell tight junctions, leading to an increase in vascular leakage and retinal edema, and disruption of the organizational structure of the neural retina. Although VEGF is considered to be a major mediator of inflammatory cell infiltration, endothelial cell proliferation and vascular leakage, other growth factors, such as PDGF, FGF, TNF, and IGF etc., are involved in these processes. Therefore, growth factor inhibitors can play a significant role in inhibiting retinal damage and the associated loss of vision upon local delivery in the eye or via oral dosing.

There is no cure for the diseases caused by ocular neovascularization and enhanced vascular permeability. The current treatment procedures of AMD include laser photocoagulation and photodynamic therapy (PDT). The effects of photocoagulation on ocular neovascularization and increased vascular permeability are achieved only through the thermal destruction of retinal cells. PDT usually requires a slow infusion of the dye, followed by application of non-thermal laser-light. Treatment usually causes the abnormal vessels to temporarily stop or decrease their leaking. PDT treatment may have to be repeated every three months up to 3 to 4 times during the first year. Potential problems associated with PDT treatment include headaches, blurring, and decreased sharpness and gaps in vision and, in 1-4% of patients, a substantial decrease in vision with partial recovery in many patients. Moreover, immediately following PDT treatment, patients must avoid direct sunlight for five (5) days to avoid sunburn. Recently, a recombinant humanized IgG monoclonal antibody fragment was approved (ranibizumab) in the US for treatment of patients with age-related macular degeneration. This drug is typically administered via intravitreal injection once a month.

Many compounds that may be considered potentially useful in treating ocular neovascularization and enhanced vascular permeability-related and other disorders, are poorly soluble in water. A poorly water soluble compound is a substance that is not soluble at a therapeutically effective concentration in an aqueous physiologically acceptable vehicle. Aqueous solubility is an important parameter in formulation development of a poorly water soluble compound. What is needed is a formulation that provides increased solubility of the compound while also providing sufficient bioavailability of the compound so as to maintain its therapeutic potential.

The present invention provides safe and effective formulations for ocular administration of poorly soluble compounds for the treatment of ocular diseases caused by endothelial cell proliferation, vascular leakage, inflammation and angiogenesis.

### SUMMARY OF THE INVENTION

The present invention overcomes these and other drawbacks of the prior art by providing compositions for treating ocular diseases due to angiogenesis and increased vascular permeability. Within one aspect of the present invention, erodible gel compositions are provided. The compositions of the invention include (a) an active agent, and (b) a suitable co-solvent, such as high molecular weight polyethylene glycol (i.e., PEG 1500 to PEG 8000) or blends of high and low molecular weight PEGs, in appropriate amount to obtain an erodible gel. The amount and molecular weight of co-solvent plays a very important role on the efficacy of the formulation upon local delivery.

A wide variety of molecules may be utilized within the scope of the present invention, especially those molecules having very low solubility. As used herein, the term “poor solubility” is used to refer to a compound having a solubility in water of less than 10 microgram/mL. The active agent for use in the compositions of the invention may be an anti-angiogenic agent, an anti-inflammatory agent, or an anti-vascular permeability agent, or any other poorly water soluble active agent useful for treating ocular disorders.

The erodible gel compositions of the present invention are preferably administered to the eye of a patient suffering from a disorder characterized by neovascularization, inflammation, or vascular permeability via posterior juxtascleral administration or intravitreal injection.

### DETAILED DESCRIPTION PREFERRED EMBODIMENTS

As noted above, the present invention provides compositions that contain an active agent for use in the treatment of ocular disorders caused by endothelial cell proliferation, enhanced vascular permeability, inflammation, or angiogenesis. The compositions of the invention are useful in preventing or inhibiting neovascularization and vascular leakage associated with such ocular disorders. In some cases, the compositions of the invention cause regression of neovascularization. Briefly, within the context of the present invention, active agents should be understood to be any molecule, either synthetic or naturally occurring, which acts to inhibit vascular growth, reduce vascular permeability, and/or decrease inflammation. In particular, the present invention provides erodible gel compositions comprising an active agent in a therapeutically effective amount, a suspending agent, and a suitable amount of a co-solvent, such as high molecular weight polyethylene glycol (i.e., PEG 1500 to PEG 8000), or blends of high and low molecular weight PEGs, in appropriate amount to obtain an erodible gel.

Polyethylene glycols (PEG) have a general chemical formula HOCH₂(CH₂OCH₂)ₙCH₂OH. They are nonvolatile, water soluble or water-miscible compounds and chemically inert, varying in molecular weight from several hundred to several thousand. They are liquids or waxy solids identified by numbers which are an approximate indication of molecular weight. PEG 400 is a liquid, while PEG 4000 is a waxy solid.

The preferred co-solvent for use in the formulations of the invention is polyethylene glycol PEG 1500 to PEG 8000. The most preferred co-solvent for use in the formulations of the present invention is PEG 1500 to PEG 3350. In certain other preferred embodiments, the gel formulations of the present invention will include blends of high and low molecular weights of polyethylene glycols to achieve better results. For example, PEG 3350 and PEG 400 in a weight ratio of 4:6 form a water-miscible erodible gel.

The co-solvent will typically be present in the formulation of the invention in an amount from 70% to 99.999%. Preferably, the compositions for the invention will contain from 90% to 99.9% co-solvent. Most preferably, the composition for intravitreal injection will contain 99% co-solvent. The composition for posterior juxtascleral, periocular and topical administration will most preferably contain 99% co-solvent.

Polyethylene glycol or blends of high and low molecular weight PEG, is identified as a key excipient for efficacious intravitreal, periocular and posterior juxtascleral erodible gel formulations.

One advantage of the present invention is that the PEG water-miscible erodible gel formulation can dissolve in water at a controlled rate, while the active agent in the formulation forms a colloidal dispersion at the site of action. This suggests that the PEG water-soluble carrier can be eliminated from the eye. At the end of use, the system does not need to be retrieved. This is a significant improvement over other existing forms of delivering active agents to the eye of a patient because it allows for fewer invasive procedures while providing superior treatment of the neovascularization, vascular permeability or other ocular disorder. The erosion can be controlled by the ratio of low and high molecular weights of PEGs. The desirable bioavailability can be achieved by controlling the rate of erosion and the rate of dissolution of the colloidal particles which is formed in-situ.

It is contemplated that any active agent that is poorly water soluble may be included in the compositions of the present invention. For example, anti-angiogenic agents, anti-inflammatory agents, or anti-vascular permeability agents are useful in the compositions of the invention.

Preferred anti-angiogenic agents include, but are not limited to, receptor tyrosine kinase inhibitors (RTKi), in particular, those having a multi-targeted receptor profile such as that described in further detail herein; angiostatic cortisenes; MMP inhibitors; integrin inhibitors; PDGF antagonists; antiproliferatives; HIF-1 inhibitors; fibroblast growth factor inhibitors; epidermal growth factor inhibitors; TIMP inhibitors; insulin-like growth factor inhibitors; TNF inhibitors; antisense oligonucleotides; etc. and prodrugs of any of the aforementioned agents. The preferred anti-angiogenic agent for use in the present invention is a multi-targeted receptor tyrosine kinase inhibitor (RTKi). Most preferred are RTKi's with multi-target binding profiles, such as N-[4-(3-amino-1H-indazol-4-yl)phenyl]-N′-(2-fluoro-5-methylphenyl)urea, having the binding profile substantially similar to that listed in Table 1. Additional multi-targeted receptor tyrosine kinase inhibitors contemplated for use in the compositions of the present invention are described in U.S. Application Serial No. 2004/0235892, incorporated herein by reference. As used herein, the term “multi-targeted receptor tyrosine kinase inhibitor” refers to a compound having a receptor binding profile exhibiting selectivity for multiple receptors shown to be important in angiogenesis, such as the profile shown in Table 1, and described in co-pending U.S. application serial number 2006/0189608, incorporated herein by reference. Most preferably, the compounds for use in the formulations of the present invention will have a receptor binding profile of KDR (VEGFR2), Tie-2 and PDGFR.

TABLE 1
Kinase Selectivity Profile of a RTK Inhibitor

| KDR | FLT1 | FLT4 | PDGFR | CSF1R | KIT | FLT3 | TIE2 | FGFR | EGFR | SRC |
|---|---|---|---|---|---|---|---|---|---|---|
| 4 | 3 | 190 | 66 | 3 | 14 | 4 | 170 | >12,500 | >50,000 | >50,000 |

All data reported as IC₅₀ values for kinase inhibition in cell-free enzymatic assays; ND denotes no data. Values determined @ 1 mM ATP.

Other agents which will be useful in the compositions and methods of the invention include anti-VEGF antibody (i.e., bevacizumab or ranibizumab); VEGF trap; siRNA molecules, or a mixture thereof, targeting at least two of the tyrosine kinase receptors having IC₅₀ values of less than 200 nM in Table 1; glucocorticoids (i.e., dexamethasone, fluoromethalone, medrysone, betamethasone, triamcinolone, triamcinolone acetonide, prednisone, prednisolone, hydrocortisone, rimexolone, and pharmaceutically acceptable salts thereof, prednicarbate, deflazacort, halomethasone, tixocortol, prednylidene (21-diethylaminoacetate), prednival, paramethasone, methylprednisolone, meprednisone, mazipredone, isoflupredone, halopredone acetate, halcinonide, formocortal, flurandrenolide, fluprednisolone, fluprednidine acetate, fluperolone acetate, fluocortolone, fluocortin butyl, fluocinonide, fluocinolone acetonide, flunisolide, flumethasone, fludrocortisone, fluclorinide, enoxolone, difluprednate, diflucortolone, diflorasone diacetate, desoximetasone (desoxymethasone), desonide, descinolone, cortivazol, corticosterone, cortisone, cloprednol, clocortolone, clobetasone, clobetasol, chloroprednisone, cafestol, budesonide, beclomethasone, amcinonide, allopregnane acetonide, alclometasone, 21-acetoxypregnenolone, tralonide, diflorasone acetate, deacylcortivazol, RU-26988, budesonide, and deacylcortivazol oxetanone); Naphthohydroquinone antibiotics (i.e., Rifamycin); and NSAIDs (i.e., nepafenac, amfenac).

The formulations described herein may be delivered topically, via intravitreal injection, and via posterior juxtascleral and periocular routes. Preferred co-solvents for use in the compositions of the present invention include ethylene glycol, propylene glycol, N-methyl pyrrolidinone, 2-pyrrolidinone, 3-pyrrolidinol, 1,4-butanediol, dimethylglycol monomethylether, diethyleneglycol monomethyl ether, solketal, glycerol, polyethylene glycol, polypropylene glycol etc.

The present inventors have discovered that certain active agents have a higher solubility in the presence of PEG than in water. For example, the compound N-[4-(3-amino-1H-indazol-4-yl)phenyl]-N′-(2-fluoro-5-methylphenyl)urea can dissolve completely and disperse in PEG 3350 and PEG 8000 when PEG melts at temperatures of about 60° C. to 95° C., and forms a wax-like pellet when cooled to room temperature. This pellet forms a colloidal like suspension when the pellet dissolves in water. Furthermore, using blends of high and low molecular weight PEGs can make erodible gel with the compound uniformly dispersed in it (see examples). This provides an injection dosage form for delivery of insoluble compounds via posterior juxtascleral or intravitreal administration, which enhances the solubility of the compounds and achieves the desired bioavailability. In the formulations of the present invention, the compound forms a solid solution in the matrix of the PEG.

In certain preferred embodiments, the formulation of the invention will further comprise a polymer that acts as a suspending agent to enhance the physical stability of the formulation. A number of polymers, such as hydroxypropyl methyl cellulose (HPMC), hydroxyethyl cellulose (HEC), methyl cellulose, Carboxymethylcellulose (CMC), carbopol, polyvinyl alcohol, polyvinyl pyrrolidone (PVP), xanthan, gum tragacanth, gum acacia, sodium alginate and its esters etc. can be used for this purpose.

The specific dose level for any particular human or animal depends upon a variety of factors, including the activity of the active compound used, the age, body weight, general health, time and frequency of administration, route of administration and the severity of the pathologic condition undergoing therapy.

The preferred gel formulations of the invention, for administration via intravitreal injection, periocular administration, posterior juxtascleral administration, topical ocular administration, may contain:
An active agent in a therapeutically effective amount;
PEG 400, PEG 3350 or PEG 8000 as a co-solvent in an effective amount to obtain an erodible gel formulation.

Certain other preferred gel formulations of the invention may contain:
An anti-angiogenic agent in a therapeutically effective amount;
A ratio of polyethylene glycol co-solvent in an amount effective to form a gel solution.

Preferred ratios of high molecular weight PEG to low molecular weight PEG for the polyethylene glycol co-solvents for use in the gel formulations of the present invention are from 7:3 to 1:8. Most preferred ratios of polyethylene glycol compounds for use in the gel formulations of the present invention are from 7 parts PEG 3350 to 3 parts PEG 400 (7:3) to 1 part PEG 3350 to 8 parts PEG 400 (1:8). The most preferred ratio for the polyethylene glycol co-solvents for use in the gel formulations of the present invention is approximately 4 parts PEG 3350 to approximately 6 parts PEG 400, as illustrated in Table 2.

The following table gives formulation of RTKi and PEG erodible gel.

TABLE 2

| | | 1% RTKi |
|---|---|---|
| Code | Gel Vehicle | PEG Gel |
| COMPONENT | % w/w | % w/w |
| RTKi | — | 1 |
| polyethylene glycol 400 | 60 | 59.4 |
| polyethylene glycol 3350 | 40 | 39.6 |

Due to the intended route of administration (IVT or PJ), it is very important that the particle size of the formulations must be small to accomplish good syringibility, as well as comfort. The prepared formulations (for IVT or PJ) exhibit excellent syringibility even when only 2 μL-10 μL of the formulation is injected in the eyes of the animals.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

Procedure for Making a RTKi-PEG 3350 Pellet

In a suitable vessel, weight and add PEG 3350 powder. Put the vessel to a 75-80° C. water bath, mix and allow PEG 3350 to melt. Add RTKi (N-[4-(3-amino-1H-indazol-4-yl)phenyl]-N′-(2-fluoro-5-methylphenyl)urea) to the PEG melt. Mix and allow RTKi to dissolve to the melt completely. Put the vessel containing RTKi-PEG melt mixture to room temperature. A hard wax pellet is formed. It is not hygroscopic.

### EXAMPLE 2

Procedure for Making RTKi-PEG Water-Miscible Erodible Gel

In a suitable vessel, weigh and add two different grades of PEG. For example, add PEG 3350 powder and PEG 400 liquid in adequate ratio. Add RTKi (N-[4-(3-amino-1H-indazol-4-yl)phenyl]-N′-(2-fluoro-5-methylphenyl)urea) raw material powder to the vessel. Then put the vessel to a 70-90° C. water bath, mix and allow PEG 3350 to melt and RTKi to dissolve completely. Allow the vessel containing RTKi-PEG melt mixture cool to room temperature. An RTKi-PEG water miscible erodible gel is formed.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

We claim:

1. An ophthalmic composition for treating ocular neovascularization, said composition comprising:
i) an active agent in an amount of from 0.01% to 30%, and
ii) a polyethylene glycol co-solvent having a molecular weight of from 1500 to 8000 or blends of high and low molecular weight PEGs;
wherein said co-solvent is present in an amount such that the composition forms a gel.

2. The ophthalmic composition of claim 1, wherein the active agent is selected from the group consisting of anti-angiogenic agents, anti-inflammatory agents, and anti-vascular permeability agents.

3. The ophthalmic composition of claim 2, wherein the active agent is an anti-angiogenic agent.

4. The ophthalmic composition of claim 3, wherein the anti-angiogenic agent is a multi-targeted receptor tyrosine kinase (RTK) inhibitor.

5. The ophthalmic composition of claim 4, wherein the RTK inhibitor is N-[4-(3-amino-1H-indazol-4-yl)phenyl]-N′-(2-fluoro-5-methylphenyl)urea.

6. The ophthalmic composition of claim 1, wherein the concentration of the active agent is from 1% to 15%.

7. The ophthalmic composition of claim 3, wherein the concentration of the anti-angiogenic agent is from 1% to 15%.

8. The ophthalmic composition of claim 1, wherein the co-solvent is PEG 3350.

9. The ophthalmic composition of claim 1, wherein the PEG co-solvent is a blend comprising PEG 400 and PEG 3350, wherein the ratio of PEG 400 to PEG 3350 is such that the composition forms a water-miscible erodible gel.

10. The ophthalmic composition of claim 9, wherein the concentration of PEG 400 in the formulation is from 30% to 90%.

11. An ophthalmic gel composition for posterior juxtasclaral administration, said composition comprising:
i) an active agent, wherein the concentration of the active agent is from 0.01% to 30%; and
ii) a polyethylene glycol co-solvent having a molecular weight of from 400 to 8000 or blends of high and low molecular weight PEGs.

12. The ophthalmic gel composition of claim 11, wherein the active agent is selected from the group consisting of anti-angiogenic agents, anti-inflammatory agents, and anti-vascular permeability agents.

13. The ophthalmic gel composition of claim 12, wherein the active agent is an anti-angiogenic agent.

14. The ophthalmic gel composition of claim 13, wherein the anti-angiogenic agent is a multi-receptor targeted receptor tyrosine kinase (RTK) inhibitor.

15. The ophthalmic gel composition of claim 11, wherein the co-solvent is PEG 3350.

16. The ophthalmic gel composition of claim 11, wherein the co-solvent is a blend of high and low moleculr weight PEGs comprising PEG 400 and PEG 3350, wherein the ratio of PEG 400 to PEG 3350 is such that the composition is forms a water-miscible erodible gel.

17. The ophthalmic gel composition of claim 16, wherein the ratio of PEG 400 to PEG 3350 is from 3:7 to 8:1.

18. The ophthalmic gel composition of claim 17, wherein the ration of PEG 400 to PEG 3350 is about 6:4.

19. A composition for intravitreal injection for the treatment of ocular neovascularization, said composition comprising:
from 0.1 to 10% of a multi-targeted receptor tyrosine kinase inhibitor;
a ratio of PEG 400 to PEG 3350 such that the composition is forms a water-miscible erodible gel.

20. The composition of claim 19, wherein the RTK inhibitor is N-[4-(3-amino-1H-indazol-4-yl)phenyl]-N′-(2-fluoro-5-methylphenyl)urea.
